(19) United States
(12) Patent Application Publication
(10) Pub. No.: US 2021/0360896 A1
(43) Pub. Date: Nov. 25, 2021

(54) AUTOMATICALLY SELF-CLEANING PET POTTY

(71) Applicant: Sebastian Waddell, Rosebery (AU)

(72) Inventor: Sebastian Waddell, Rosebery (AU)

(21) Appl. No.: 17/326,055

(22) Filed: May 20, 2021

(30) Foreign Application Priority Data
May 22, 2020 (AU) 2020901664

(51) Int. Cl. A01K 1/01

(52) U.S. Cl. CPC A01K 1/0135

(57) ABSTRACT

The pet potty has a waste surface loop, rollers within the potty for rotating the waste surface loop and a water jet for hosing off the waste surface loop as it rotates on the rollers within the potty. The characterising feature of the invention is the that the potty includes a waste strainer positioned underneath the water jets for collecting objects which cannot be disintegrated by the water jets so that those objects can be conveniently removed by the waste strainer from the potty.

### BACKGROUND OF THE INVENTION

### (1) Technical Field

The present invention relates to the pet product industry and, more particularly to an automatically self-cleaning pet potty.

### (2) Background Art

Cleaning up stool and urine of pets is an onerous part of pet ownership. Pets often prefer to defecate outside and on grass. However, it is not always convenient to take pets on walks to defecate. People are often not inclined to leave doors open in their homes overnight so that pets can defecate outside. Moreover, many people live in units where the outdoors is not accessible for pets to defecate.

People sometimes put newspaper on the floor and train their pets to defecate on it. Some people use kitty litter boxes. Other people use turf trays which allow the stool to be collected from the turf and urine to drain through the turf into a bowl. However, in all these cases, the stools have to be manually collected and deposited in toilets or in stool bags.

In the case of the turf trays, urine crystalizes on the turf patches which causes a foul odour. The turf patches have to be replaced regularly at a cost of around US $20 each. In many cases, people abandon the ongoing use of the turf trays because of the expense of replacing the turf. Bacteria stick to the turf surface and are difficult to eradicate from it.

Artificial turf can be used in these trays, but it needs to be taken off the tray and hosed down regularly. The hosing down can be either indoors with a shower hose or outdoors with a garden hose. This can be a messier and less hygienic job than simply picking up the stool.

Attempts in the prior art have been made at automatically self-cleaning pet potties. However, the structure of these prior art potties is such that the stools are not effectively cleaned from the defecation surface and ejected from the potty, which leaves a foul odour and a health hazard. Moreover, hard objects such as bones in dog stool, or particularly hard nuggets of stool clog the entire self-cleaning system. It is more onerous to clean stool out of a clogged self-cleaning potty than it is to simply pick up the stool up from the ground. The object of the present invention is to overcome the aforementioned problems with the prior art.

### SUMMARY OF THE INVENTION

According to the present invention, there is provided a pet potty comprising:
(a) a waste surface loop;
(b) rollers within the potty for rotating the waste surface loop; and
(c) a water jet for hosing off the waste surface loop as it rotates on the rollers within the potty,
   wherein the potty includes a waste strainer positioned underneath the water jet for collecting objects which cannot be disintegrated by the water jet so that those objects can be conveniently removed by the waste strainer from the potty.

The pet potty may include an electronic waste analyser. The electronic waste analyser is preferably adapted to detect the presence of parasites in the waste. The electronic waste analyser is preferably powered by a solar panel on the potty. The pet potty may include a sanitiser unit adapted to hold sanitising tablets which dissolve into the water travelling through the water jets to hose off the waste surface loop. The sanitiser unit is preferably located internally within the pet potty.

Preferably, the rollers are powered hydraulically rather than electrically. The pet potty preferably connected to a water hose with a timer unit for activating the rollers at set intervals.

### BRIEF DESCRIPTION OF THE DRAWINGS

Embodiments of the invention will be described with reference to the following drawings, in which:

FIG. 1 is a perspective view of the pet potty according to the present invention.

FIG. 2 is the pet potty of FIG. 1 with the top cover removed to expose the water jets used to clean the defecation surface of the potty and disintegrate the stool.

FIG. 3 is a side section close up view of the potty of FIG. 1 showing the sanitiser and the waste analyser in the pet potty.

FIG. 4 is a rear view of the pet potty of FIG. 1.

FIG. 5 is a front view of the pet potty of FIG. 1, with the water jets flowing.

FIG. 6 is an exploded view of the pet potty of FIG. 1.

FIG. 7 is an exploded view of the gear system which drives the defecation surface loop of the pet potty of FIG. 1.

FIG. 8 is a side section view of the pet potty of FIG. 1.

FIG. 9 is a schematic illustration of the waste analyser system of the pet potty of FIG. 1.

### DETAILED DESCRIPTION OF THE INVENTION

The figures show an embodiment of a pet potty 10 according to the present invention for a dog 12. The pet potty 10 has a waste surface loop 14. The waste surface loop 14 may have a texture, such as a low pile artificial grass to attract a pet to the potty 10. However, the waste surface loop 14 may also be smooth to facilitate cleaning.

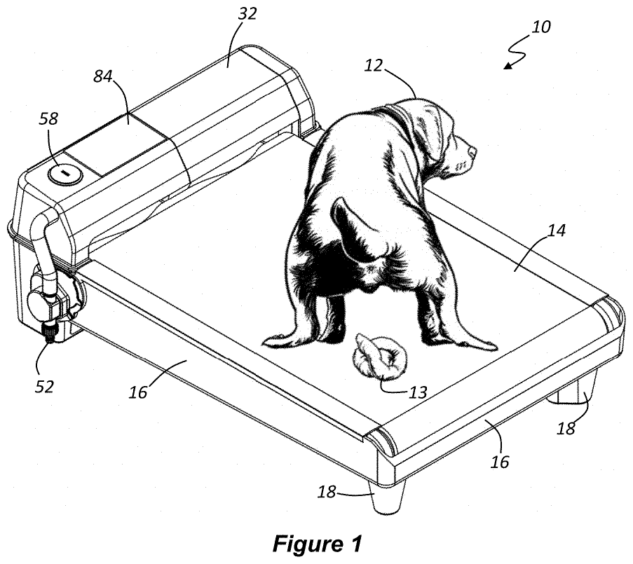

The potty 10 rests on a base 16 (see FIG. 1). The base 16 has legs 18 to make it easier to clean underneath the potty 10. The legs 18 have rubber protectors 20 (see FIGS. 3, 4 and 5) underneath them in to prevent the legs 18 from scratching the floor on which the potty 10 is placed. Another reason that the legs 18 have protectors is so that if a person drags the potty 10 across the floor and legs sustain a crack or a hole, then the legs 18 could become a point at which the potty 10 leaks liquid. The protectors 20 help to prevent the overall potty 10 from leaking. The legs 18 have a cap (not shown) so that they are sealed off to allow liquid to drain across internal side of the base 16 toward the waste catch area 22.

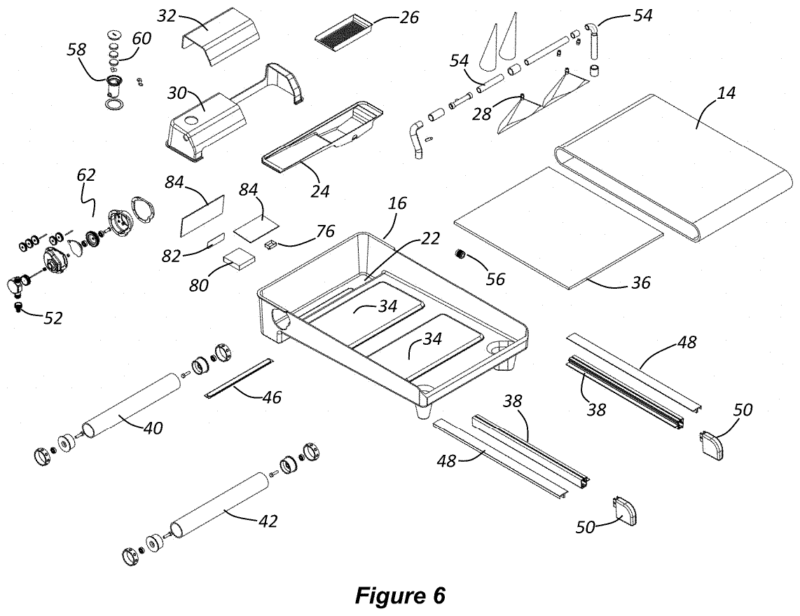

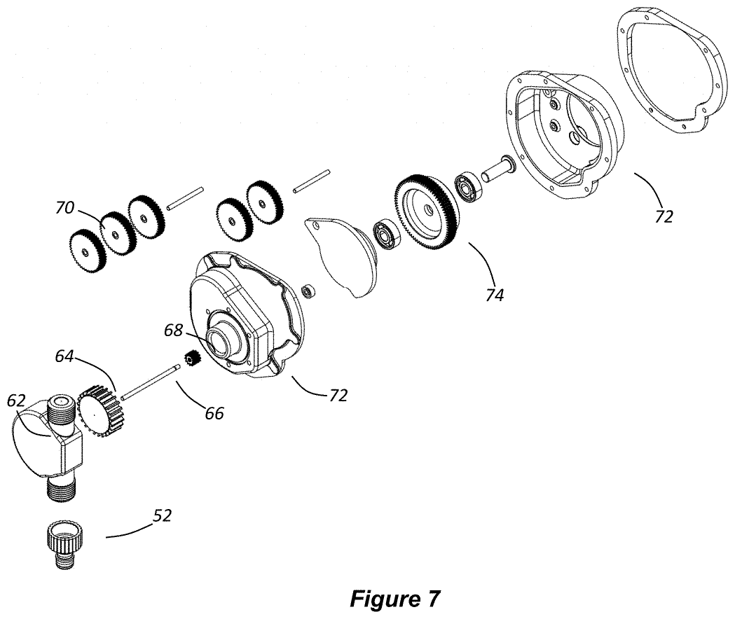

Referring to FIG. 6, the waste catch area 22 contains a waste catcher 24 which holds a waste strainer 26. The waste strainer 26 is positioned directly under water jets 28 designed to disintegrate the stool 13 (shown in FIG. 1) and rinse the surface of the waste surface loop 14. The waste strainer 26 makes a substantial contribution to the working of the invention because it catches particularly hard nuggets of stool and exposes them directly at the jets 28 so that they can have water passes through them to dissolve and disintegrate them. The waste strainer 26 also catches hard pieces of bone which may be present in the dog stool. The waste strainer 26 can be easily removed from the waste catcher 24 so that the contents of the waste strainer 26 can be tipped into a trash bin. Hard nuggets and bone fragments tend to block and clog prior art designs of automatically self-cleaning pet potties. The inventor found that it was more trouble to clean a prior art self-cleaning pet potty 10 than it was to simply pick up the stool 13 from the ground. Handling dog stool is a particularly revolting task and any dealings with dog stool need to be minimised. Fortunately, the present invention overcomes this significant problem with the prior art.

The base 16 has a cover 30 with a flap door 32 which can swing open to reveal the waste strainer 26. The base 16 also has mounds 34 which strengthen the base 16 and minimise its flexing and warping. The mounds 34 are upwardly raised so that liquid drains off them and downward the waste catch area 22.

The waste surface loop 14 is supported by a bridge 36 which rests on the base 16. The bridge 36 supports the weight of the dog 12. The bridge 36 rests on rails 38 on the inside side wall of the base 16.

The waste surface loop 14 is driven by a lead roller 40. The surface loop 14 is also guided by an idler roller 42. The rollers 40 and 42 are actuated by cogs. The cogs 40 and 42 have knobs 44 which engage with holes in the surface loop 14. Without the knobs 44, the surface loop 14 can slip around the rollers 40 and 42 so that it does not effectively rotate within the potty 10. The surface loop 14 is prone to slipping on the rollers 40 and 42 when the loop 14 becomes wet. This is a problem which is not resolved in the prior art. The surface loop 14 can also expand when heated by the sun. The inventor identified that a slack loop is harder to spin than a tight loop. However, the knobs 44 on the cogs 40 and 42 can compensate for this problem by engaging with corresponding holes (not shown) in the loop 14 irrespective of the rigidity of the loop 14.

The waste catcher 24 has a scraper 46 which abuts the waste surface loop 14 which scrapes stool from the surface loop 14 at it rotates around the rollers 40 and 42.

The edges of the surface loop 14 are covered by side guards 48 and end guards 50 which prevent paws and fingers from being pinched between the surface loop 14 and the base 16 of the pet potty 10.

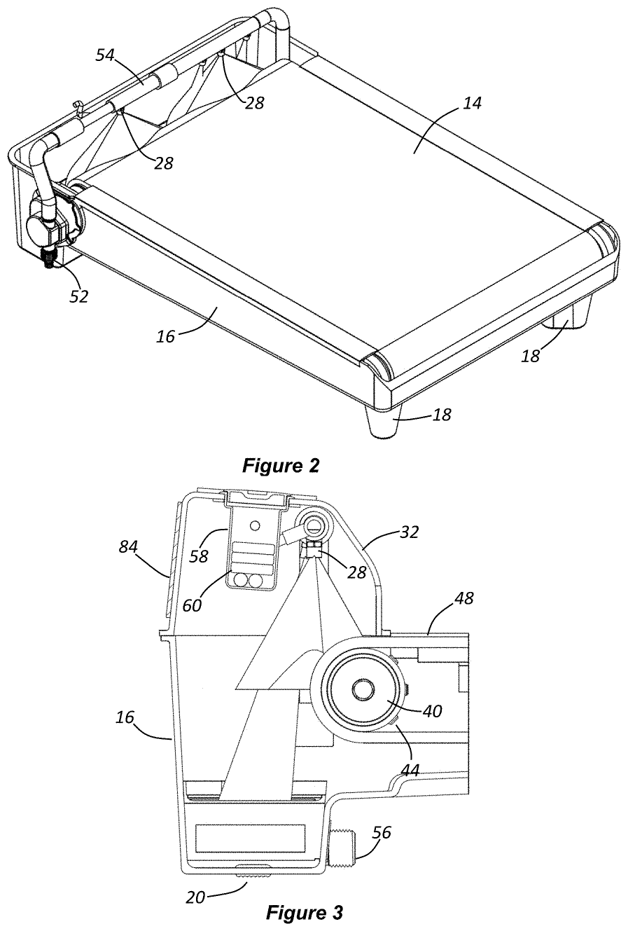

Referring to FIGS. 2 and 3, the potty 10 has a water input 52 a water channel 54 and an output 56 (shown in FIG. 3) which is connected by a hose (not shown) to an external drain (not shown).

The water channel 54 within the potty 10 passes through a sanitiser unit 58. The sanitiser unit 58 contains a stack of sanitising tablets 60 which are adapted to dissolve a sanitising agent in the water. The sanitising agent minimises the smell and can act as a disinfectant agent for bacteria on the waste surface loop 14.

Prior art versions of self-cleaning pet potties used disinfection liquid in the water channel. However, the present inventor identified that a substantial amount of liquid is taken up by the water channel so that the disinfectant liquid needs to be replaced too frequently. As a result, the prior art potties are too costly to operate with disinfectant liquid and too laborious to maintain. Another problem with prior art self-cleaning potties is that the disinfection liquid container is located outside the potty 10, where the poisonous disinfectant liquid is within reach of children and can potentially be ingested by pets. The present invention solves this problem by having sanitising tablets 60 located within the potty 10.

The water coming into the potty 10 travels through a turbine 62 that drives impeller 64 which is supported by a pin 66 which is connected to a small gear 68 that engages stacked gears 70 within a housing 72. The stacked gears 70 engage with a large gear 74 in the housing 72 which then engages with the driving roller 40.

The stacked gears 70 reduce the rpm (rotations per minute) of the waste surface loop 14 to a reasonable rate, otherwise the waste can be flung from the potty 10 because the loop 14 turns at such a fast rate. The flow of the water turns the loop 14. This particular embodiment of the present invention is hydraulically powered, rather than electrically powered. The user turns an external tap (not shown) on in order to make the waste surface loop 14 turn and to disintegrate the stool 13.

The user can set an automatic timer to run the hose and clean the potty 10. Allowing the user to run a timer allows them to self-determine the appropriate running time for the potty 10 for their particular pet. Some animals defecate larger amounts than others, or more frequently than other animals. Internet connected timers can be used with the present invention in order to remotely change the frequency and timing of the pet potty 10.

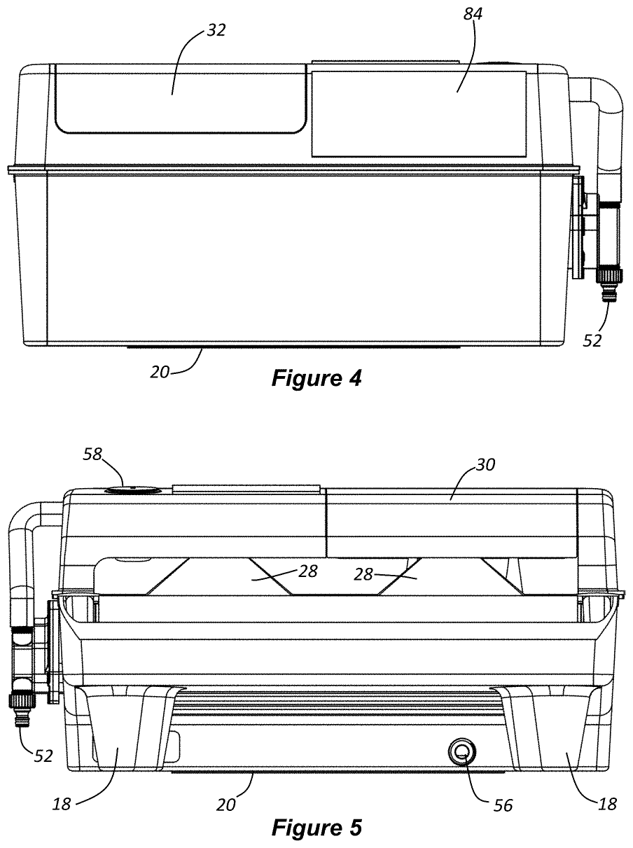

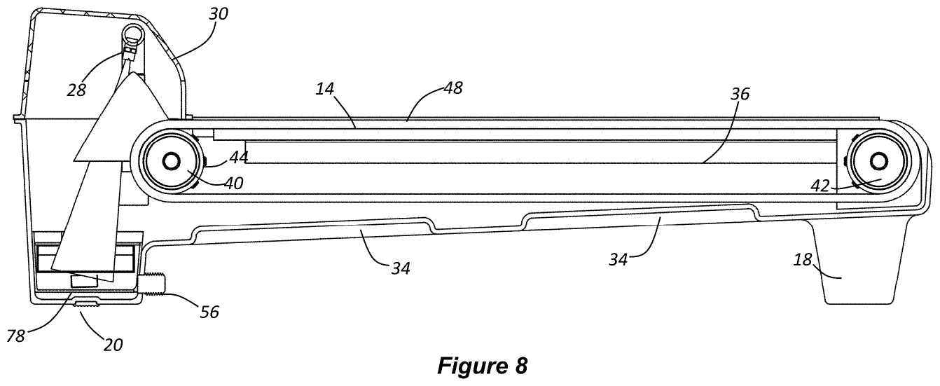

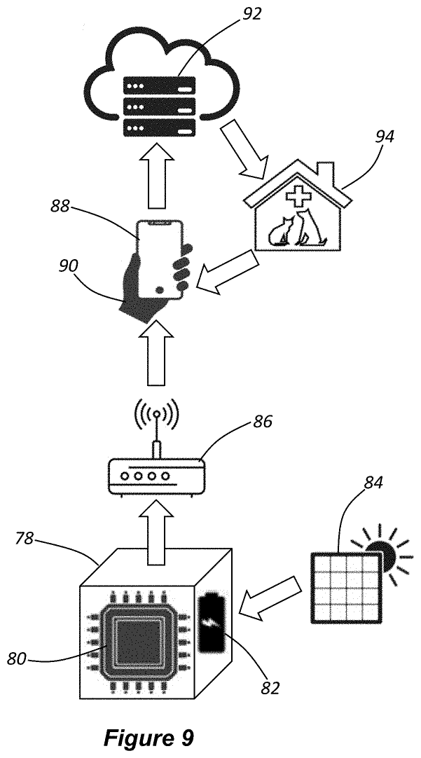

The pet potty 10 also includes a waste analyser 76. The operation of the waste analyser 76 is shown in FIG. 9. The waste analyser 76 includes a sensor unit 78 which can be seen in FIGS. 3, 6 and 8. The sensor unit 78 is adapted to perform a chemical analysis of the stool 13. The chemical analysis performed by the sensor unit 78 could reveal the presence of ailments, such as parasites, bacteria, microbes and other adverse biomarkers. Early detection of ailments can improve the health of the pet. The waste analysis data is computed by an onboard processor 80 which is powered by a battery 82 that is charged by a solar panel 84 on the cover of the pet potty 10 (see FIG. 4). The processor 80 is adapted to be connected to Wi-Fi router 86 so that it can transmit the data to the cell phone 88 of a user 90 running a software application connected to a server 92 of the present invention. The software application can use the cell phone 88 of the user 90 to transmit the data to the server 92 for analysis and storage. The data can also be transmitted from the server 92 to a nominated veterinarian 94 who can interpret the data for the user 90.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A pet potty comprising:
(a) a waste surface loop;
(b) rollers within the potty for rotating the waste surface loop;
(c) a water jet for hosing off the waste surface loop as it rotates on the rollers within the potty; and
(d) a waste strainer positioned underneath the water jet for collecting objects which cannot be disintegrated by the water jet, enabling the objects to be conveniently removed by the waste strainer from the potty.

2. The pet potty of claim 1, wherein the pet potty further includes an electronic waste analyser.

3. The pet potty of claim 2, wherein the electronic waste analyser is adapted to detect the presence of parasites in waste.

4. The pet potty of claim 2, wherein the electronic waste analyser is powered by a solar panel on the potty.

5. The pet potty of claim 1, wherein the pet potty further includes a sanitiser unit adapted to hold sanitising tablets which dissolve into water travelling through the water jets for hosing off the waste surface loop.

6. The pet potty of claim 5, wherein the sanitiser unit is located internally within the pet potty.
